(19) United States
(12) Patent Application Publication
(10) Pub. No.: US 2024/0272108 A1
(43) Pub. Date: Aug. 15, 2024

(54) NANOPARTICLES FOR CHEMIRESISTOR SENSORS

(71) Applicant: NANOSCENT LTD., Misghav (IL)

(72) Inventors: Katya KAPILOV, Karmiel (IL); Tali BOUTA BEN SHABAT, Nesher (IL); Maayan HIRSCH, Haifa (IL); Sima Simha ISRAEL, Atsmon Segev (IL)

(73) Assignee: NANOSCENT LTD., Misghav (IL)

(21) Appl. No.: 18/441,465

(22) Filed: Feb. 14, 2024

Related U.S. Application Data
(60) Provisional application No. 63/445,323, filed on Feb. 14, 2023.

(51) Int. Cl. G01N 27/12; G01N 33/00

(52) U.S. Cl. CPC G01N 27/127; G01N 33/0027

(57) ABSTRACT

A nanoparticle characterized by sensitivity to an analyte of interest and comprising a conductive core in contact with a plurality of first ligands and of second ligands bound to the conductive core is disclosed. Additionally, a chemiresistor sensor comprising the nanoparticles of the invention and a method of using thereof such as for detection of an analyte of interest in a gaseous sample are disclosed.

### CROSS REFERENCE TO RELATED APPLICATIONS

This application is a US application which claims the benefit of priority of U.S. Provisional Application No. 63/445,323, filed on Feb. 14, 2023. The contents of the above applications are all incorporated by reference as if fully set forth herein in their entirety.

### FIELD OF THE INVENTION

The present invention generally relates to chemiresistor sensors. More particularly, the present invention relates to nanoparticle for chemiresistor sensors.

### BACKGROUND OF THE INVENTION

Chemiresistor sensors are sensors that can detect the presence of volatile compound (VCs). A chemiresistor sensor includes a material or structure that changes its electrical resistance in response to changes in the nearby chemical environment, for example, due to the presence of VCs. Commercial chemiresistor sensors for sensing VCs include a sensing element made from one of: carbon nanotubes, graphene, carbon nanoparticles, conductive polymers and the like. These chemiresistor sensors are sensitive to cleaning and regeneration cycles which are required after each measurement, due to the nonuniformity nature of the sensor's material. Another optional sensor includes metallic nanoparticles cores coated with organic ligands. The organic ligands are bonded with the surface of the metallic core at one end and are configured to be weakly bonded (e.g., interact) to a VC at the other end. The most suitable and widely used cores are nanoparticles of: Au, Pt, Pd Ag and further also alloys consisting of Ni, Co, Cu, Al, Au/Ag, Au/Cu, Au/Ag/Cu, Au/Pt, Au/Pd, Au/Ag/Cu/Pd, Pt/Rh, Ni/Co, and Pt/Ni/Fe.

The most common type of organic ligands that can form a bond with the surface of a metallic particle having one of the above listed metallic cores are thiols (sulfides). Exemplary thiols that can be bonded with the metallic cores include alkylthiols with C3-C24 chains, co-functionalized alkanethiolates, arenethiolate, (3-mercaptopropyl) tri-methyloxysilane, dialkyl disulfides, xanthates, oligonucleotides, polynucleotides, peptides, proteins, enzymes, polysaccharides, and phospholipids. These thiols form relatively stable bonds in comparison with other organic ligands, however they are not stable enough and undergo dissociation over time.

The sensitivity of the chemiresistor sensor results from the chemical and physical properties of both the VC and the sensors' ligands. Specific types of VCs of interest may include volatile organic compounds (VOC) such as Aldehydes and Alkanes that can be indicative of the presence of viruses or other pathogens in an air sample. In addition, there might be a particular interest for a highly sensitive and selective detection of VCs such as inorganic oxides, and/or additional environmental pollutants.

The sensitivity of common chemiresistor sensor is affected by the relative humidity, the higher the humidity the less selectivity. Since an air sample usually includes significant amount of water vapors, along with a large variety of gases having a similar chemical structure to the analytes of interest (e.g., N₂, O₂, etc.), there is a need for a chemiresistor sensor with increased sensitivity and/or specificity to one or more VCs and/or VOCs of interest such as alkanes (C6 and higher alkanes), aldehydes, chlorinated alkanes, alcohols, ammonia, H₂S and/or NOₓ species.

### SUMMARY OF THE INVENTION

In one aspect, there is provided a composition comprising a plurality of modified noble metal nanoparticles, wherein each of the plurality of modified noble metal nanoparticles comprises a noble metal nanoparticle bound to a first ligand and to a second ligand, wherein: the first ligand and the second ligand are assembled to form a shell on top of the noble metal nanoparticle; the first ligand is a linear or branched C1-C10 mercaptoalkyl, or a linear or branched C1-C10 mercaptoalkyl-aryl; the second ligand is represented by Formula:

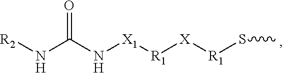

wherein a wavy bond represents H or an attachment point to the noble metal nanoparticle; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom; and wherein R₂ represents H, or an optionally substituted linear or branched alkyl.

In some embodiments, a total number of carbon atoms within the second ligand is between 6 and 30.

In some embodiments, a molar ratio between the first ligand and the second ligand within the composition is between 5:95 and 95:5.

In some embodiments, the modified noble metal nanoparticle is characterized by sensitivity to an analyte of interest.

In some embodiments, the analyte of interest comprises a volatile organic compound (VOC), water, nitrogen oxide (NOₓ), CO₂, ammonia, urea, H₂S, H₂, O₂, CO, sulfonated compounds, halogenated compounds, silane or any combination thereof.

In some embodiments, the VOC is selected from an optionally unsaturated C1-C20 aldehyde, an optionally unsaturated C1-C20 ketone, an optionally unsaturated C1-C20 alkane, a chlorinated alkane an aromatic compound, carboxylic acid, ester, ether, lactone, alcohol, phenol-based compounds, or any combination thereof.

In some embodiments, a total number of carbon atoms within the second ligand is between 10 and 30.

In some embodiments, the plurality of modified noble metal nanoparticles is bound to a substrate.

In some embodiments, the composition is characterized by a porosity between 5 and 90%.

In another aspect, there is provided a method for synthesizing the modified noble metal nanoparticle of the invention, the method comprises reacting a noble metal nanoparticle bound to the first ligand with a compound of Formula:

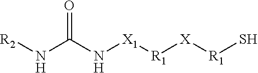

under suitable conditions, thereby obtaining the modified noble metal nanoparticle; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom; and wherein R₂ represents an optionally substituted linear or branched alkyl.

In some embodiments, the conditions comprise a temperature between −20 and 60° C.

In some embodiments, the conditions comprise a polar organic solvent with a relative polarity of at least 0.15.

In some embodiments, the conditions comprise a reaction time from 0.5 to 48 h.

In some embodiments, the noble metal nanoparticle is bound to a substrate.

In some embodiments, the contacting comprises a molar ratio between the compound and the first ligand of between 2:1 and 1000:1.

In some embodiments, the method further comprises a drying.

In another aspect, there is provided a chemiresistor sensor comprising: at least two electrodes; and a sensing element electrically connected to the two electrodes and comprising a structure made from the composition of the invention.

In some embodiments, the distribution of the modified noble metal nanoparticles within the structure is such that the entire structure is electrically conductive.

In some embodiments, the modified noble metal nanoparticles are bound to a substrate comprising a plurality of electrodes.

In some embodiments, the modified noble metal nanoparticles are in a form of a substantially continuous layer on top of the substrate.

In some embodiments, the sensor is configured for selective detection of group of analytes of interest within a gaseous sample by reducing the response to other group of analyte, and wherein the sensor is characterized by a limit of detection (LOD) ranging between 0.01 ppb and 500 ppm.

In some embodiments, the sensor is operable at a relative humidity of up to 90%. In another aspect, there is provided a method for detection of an analyte of interest in a gaseous sample, comprising: a. exposing the chemiresistor sensor of the invention to the gaseous sample. b. providing electricity to the sensor, so as to obtain a plurality of values generated by the sensor; and c. analyzing the values thereby determining the presence of the analyte of interest within the sample, wherein the values comprise conductivity values, capacitance values, impedance values, or any combination thereof.

In some embodiments, the analyte of interest comprises a volatile organic compound (VOC), nitrogen oxide (NOₓ), CO₂, ammonia, H₂S, or any combination thereof.

In some embodiments, the analyzing step further comprises determining a concentration of the analyte of interest within the sample.

In some embodiments, a concentration of the analyte of interest within the sample is between 0.1 ppb and 500 ppm.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of a chemiresistor sensor according to some embodiments of the invention.

FIG. 2 is an illustration of a particle for chemiresistor sensor according to some embodiments of the invention.

FIGS. 3A-3B is a flow chart of the method of manufacturing the modified noble metal particles, 3A. manufacturing the particles and depositing them on the substrate 3B. manufacturing the particles directly on the substrate.

FIGS. 4A-4C are graphs presenting the response of the sensor to water vapor 4A. sensor response to octanal vs. humidity, 4B. sensor response to humidity, and 4C. humidity sensor.

FIG. 5 are graphs presenting the sensor response to octanal and 5% relative humidity, at three different temperatures the ligand exchange reaction was performed at (0, 20 and 30°).

FIGS. 6A-6C are graphs presenting the sensor response to 6A. acetone, 6B. octanal and 6C. 5% relative humidity.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

### Opening Statement

The invention in some embodiments thereof is based on a surprising finding that a chemiresistor sensor comprising a sensing element composed inter alia of noble metal nanoparticles comprising covalently bound to a first ligand and to a second ligand, wherein the first ligand and the second ligand assemble to form a shell on top noble metal nanoparticles obtaining the modified noble metal nanoparticles of the innovation. The modified noble metal nanoparticles exhibited an unexpected selectivity and/or sensitivity to numerous VC and/or VOCs of interest. Furthermore, the inventors found that the modified noble metal nanoparticles of the invention comprising the ligands disclosed herein are characterized by practically the same zero response to varying relative humidity, so that the sensor of the invention may be used inter alia as specific sensor for VOCs without being affected by level of humidity.

A chemiresistor sensor based on the gold nanoparticles of the invention, has been successfully implemented by the inventors for detection of aldehydes, aromatic compounds, alkanes and VCs such as NOₓ, CO₂, and water vapor in a gaseous sample. Some of the nanoparticles disclosed herein, were found to be appropriate for highly selective sensing of ketones (e.g., acetone) and aldehydes (such as Decanal, Nonanal, Octanal, Heptanal and Hexanal) at sub ppb-level (about 0.1 ppb or even less).

### The Figures

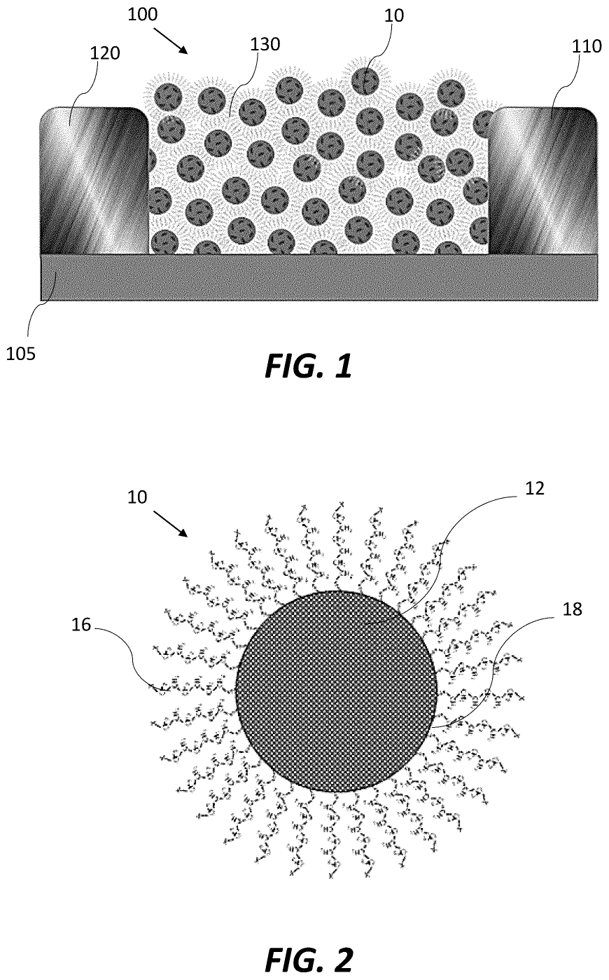

Reference is now made to FIG. 1 which is an illustration of a chemiresistor sensor according to some embodiments of the invention. A chemiresistor sensor 100 may include: two electrodes 110 and 120 and a sensing element 130 connected to electrodes 110 and 120 and comprising a deposited structure made from the particles 10 disclosed herein below. The deposited structure may be printed, sprayed, or applied on a substrate 105, using any known deposition method. Substrate 105 may be any substrate known in the art of chemiresistor sensors. Chemiresistor sensor 100 may be included in a scent recorder. Electrodes 110 and 120 may be connected to a processor (e.g., a chip) that may detect changes in the electrical resistance of sensing element 130 in response to changes in the nearby chemical environment, for example, due to the presence of VCs of interest.

In some embodiments, sensing element 130 may include a plurality of particles 10. Particles 10 may be mixed with a carrier solvent and printed/deposited in order to form sensing element 130. The carrier solvent may be evaporated prior to the use of sensing element 130.

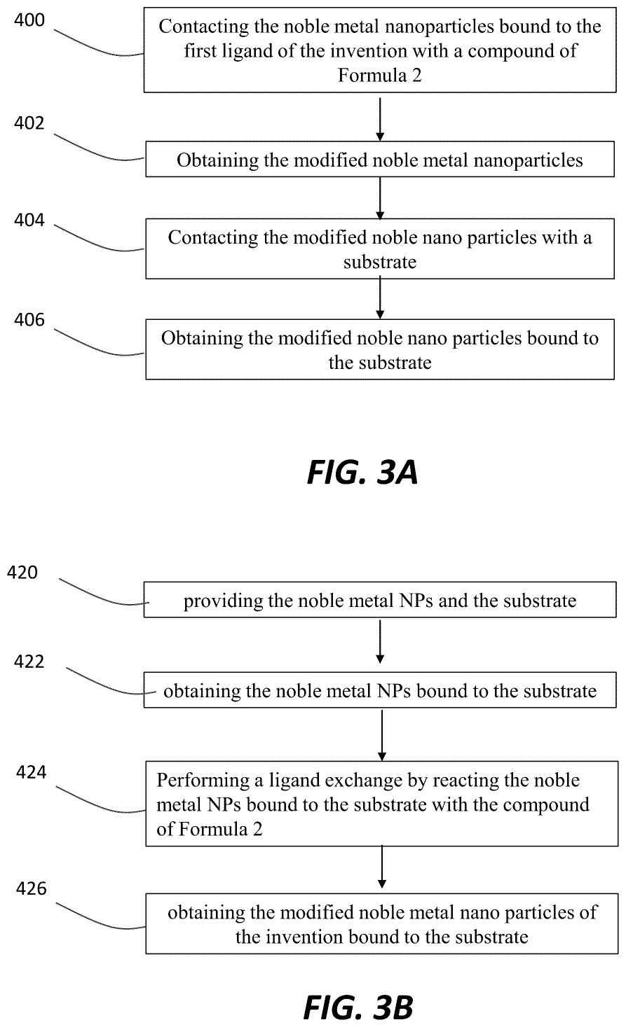

Reference is now made to FIG. 2 which is an illustration of a modified noble metal nanoparticle 10 according to some embodiment of the invention. Nanoparticle 10 includes a conductive core made of a noble metal, 12 and a plurality of the first and second ligands 16 bonded to the noble metal core 12.

In some embodiments, the modified noble metal nanoparticle of the invention is non-uniformly shaped. In some embodiments, a plurality of the modified noble metal nanoparticles of the invention is devoid of a defined shape (e.g., the particles have a random shape). In some embodiments, the modified noble metal nanoparticle of the invention is characterized by substantially spherical shape, elliptical shape, and/or a cylindrical shape. In some embodiments, the shape of the modified noble metal nanoparticle is substantially predefined by the shape of the conductive core. In some embodiments, the modified noble metal nanoparticle is a modified Au nanoparticle.

### Core

Conductive core 12 comprises or consist essentially of a noble metal (i.e., a noble metal in an elemental state). For example, conductive core 12 may include: one or more noble metal(s), in an elemental state or in an oxidized state. In some embodiments, the conductive core 12 is or comprises Au, and further also Au alloys such as but not limited to Au/Ag, Au/Cu, Au/Ag/Cu, Au/Pt, Au/Pd, Au/Ag/Cu/Pd, and any combination thereof.

In some embodiments, the noble metal is capable of forming a complex with a thiol group. In some embodiments, the noble metal is selected from but is not limited to Au, Ru, Re, Rh, Os, Ir, Pd, Pt, Hg, As, Cu, Ag, including any alloy, any salt or any oxide thereof.

In some embodiments, the noble metal conductive core is characterized by resistivity of less than 10⁻¹ Ωcm, less than 10⁻² Ωcm, less than 10⁻³ Ωcm, less than 10⁻⁴ Ωcm, less than 10⁻⁵ Ωcm, less than 10⁻⁸ Ωcm, less than 10⁻¹⁰ Ωcm, less than 10⁻¹² Ωcm, including any range between, when measured at 20° C.

In some embodiments, the noble metal within the conductive core is in an elemental state, or in an oxidized state (e.g., a metal oxide and/or a metal salt). In some embodiments, at least 90%, at least 93%, at least 95%, at least 97%, at least 99.9%, or 100% including any range between, by weight of the noble metal within the conductive core is in a form of a metal oxide. In some embodiments, at least 90%, at least 93%, at least 95%, at least 97%, at least 99.9%, or 100% including any range between, by weight of the metal within the conductive core is in an elemental state. As used herein, the term “elemental state” refers to zero oxidation state of an atom.

In some embodiments, at least 60%, at least 70%, at least 80%, at least 90%, at least 93%, at least 95%, at least 97%, at least 99.9%, or 100% including any range between, by weight of the conductive core is composed of the conductive metal (i.e., Au).

In some embodiments, the conductive metal (I.e., Au) is in a crystalline state. In some embodiments, at least 80%, at least 90%, at least 93%, at least 95%, at least 97%, at least 99.9%, or 100% including any range between, by weight of the conductive metal is in a crystalline state.

In some embodiments, the noble metal is in an amorphous state. In some embodiments, at least 80%, at least 90%, at least 93%, at least 95%, at least 97%, at least 99.9%, or 100% including any range between, by weight of the noble metal is in an amorphous state.

In some embodiments, the conductive core comprises an inner portion and an outer portion facing the ambient. In some embodiments, the outer portion is bound to the ligands of the invention. In some embodiments, the inner portion comprises about 97%, about 98%, about 99%, about 99.9%, about 99.99%, about 99.999% of the entire weight and/or entire volume of the conductive core. In some embodiments, the outer portion refers to the outer surface of the conductive core. In some embodiments, the outer portion is in a form of a layer. In some embodiments, the outer portion comprises of one or more (e.g., 2, 3, 5, or 10) atomic layer(s).

In some embodiments, the inner portion and/or the outer portion of the conductive core is substantially devoid of an organic compound. In some embodiments, the inner portion and/or the outer portion is substantially devoid of a non-conductive metal and/or a non-conductive metal oxide, or a non-conductive metal salt. In some embodiments, the entire conductive core is substantially devoid of: an organic compound, a non-conductive metal, a non-conductive metal oxide, or a non-conductive metal salt including any combination thereof.

In some embodiments, the conductive core is or comprises Au.

In some embodiments, the average cross section size of conductive core 12 may be of at most 100 nm. In some embodiments, the average cross section size of conductive core 12 may be of at most 80 nm. In some embodiments, the average cross section size of conductive core 12 may be of at most 70 nm. In some embodiments, the average cross section size of conductive core 12 may be of at most 60 nm. In some embodiments, the average cross section size of conductive core 12 may be of at most 50 nm. In some embodiments, the average cross section size of conductive core 12 may be of at most 40 nm. In some embodiments, the average cross section size of conductive core 12 may be of at most 30 nm. In some embodiments, the average cross section size of conductive core 12 may be of at most 20 nm. In some embodiments, the average cross section size of conductive core 12 may be of at most 10 nm. In some embodiments, the average cross section size of conductive core 12 may be of at most 5 nm, for example, 1 nm, 2 nm, 3 nm, etc.

In some embodiments, the average cross section size of the conductive core of the invention is between 1 and 100 nm, between 1 and 10 nm, between 10 and 50 nm, between 50 and 100 nm, including any range between. In some embodiments, the average cross section size of the conductive core of the invention is between 1 and 20 nm, between 1 and 10 nm, between 10 and 15 nm, between 15 and 20 nm, between 1 and 5 nm, between 1 and 3 nm, between 3 and 5 nm, between 5 and 7 nm, between 7 and 10 nm, including any range between.

In some embodiments, the modified noble metal nanoparticles of the invention are characterized by a particle size in a range between 1 and 100 nm, between 1 and 10 nm, between 10 and 50 nm, between 50 and 100 nm, including any range between. In some embodiments, the modified noble metal nanoparticles of the invention are characterized by a particle size in a range between 1 and 5 nm, between 1 and 3 nm, between 3 and 5 nm, between 5 and 7 nm, between 7 and 10 nm, including any range between.

In some embodiments, the term “particle size” refers to average cross section size of the noble metal nanoparticles.

In some embodiments, the term “average cross section size” may refer to either the average of at least e.g., 70%, 80%, 90%, or 95% of the modified noble metal nanoparticles, or in some embodiments, to the median size of the plurality of modified noble metal nanoparticles. In some embodiments, the term “average cross section size” refers to a number average of the plurality of modified noble metal nanoparticles. In some embodiments, the term “average cross section size” may refer to an average diameter of substantially spherical modified noble metal nanoparticles.

### Shell

In some embodiments, the modified noble metal nanoparticle of the invention comprises the noble metal core, wherein the outer portion of the noble metal core is bound to a plurality of ligands of the invention.

In some embodiments, ligands 16 are assembled to form a shell on top of core 12. In some embodiments, ligands 16 are selected to make modified noble metal nanoparticle 10 characterized by sensitivity to the analyte of interest, wherein the analyte of interest is as described herein. In some embodiments, ligands 16 are selected to make the modified noble metal nanoparticle 10 characterized by binding affinity for the analyte of interest, wherein the analyte of interest is as described herein. In some embodiments, the binding moiety is thiol, or (—S—) including any salt thereof.

In some embodiments, the ligands are covalently bound to the outer portion (or outer surface) of the conductive core, to form a shell. In some embodiments, the ligands are bound to the conductive core via the binding moiety (or via a derivative thereof). In some embodiments, each ligand is bound to the Au atom of the conductive core via a covalent bond (including inter alia a coordinative bond) and/or a non-covalent bond (such as hydrogen bonds, electrostatic interactions, Van-der-Waals bonds, dipol-dipol interactions, etc.). In some embodiments, each ligand is stably complexed by the metal (e.g., the ligand forms a coordinative bond with the Au atom). In some embodiments, the ligands are chemisorbed and/or physisorbed to the conductive core. In some embodiments, the ligands are stably bound to the conductive core. In some embodiments, the term “stable” refers to the chemical stability (e.g., substantially devoid of bond cleavage) of the bond under ambient conditions (a temperature of less than 100° C., normal pressure or vacuum, and optionally ambient atmosphere), for a time period of between 1 day and 1 year including any range between.

In some embodiments, the ligands are bound to the outer portion of the conductive core, so as to form of a self-assembled monolayer (SAM). In some embodiments, the plurality of ligands are in a form of SAM on top the conductive core, thus forming a shell. In some embodiments, the shell is in a form of a layer. In some embodiments, the shell is in a form of a consecutive layer, optionally having a substantially uniform thickness. In some embodiments, the shell substantially encloses the conductive core.

In some embodiments, the shell is substantially devoid of a metal. In some embodiments, the shell is substantially devoid of an additional molecule other than the ligands. In some embodiments, the shell comprises trace level impurities such as an organic solvent and/or one or more impurities (e.g., aliphatic molecules, and/or unbound ligands).

In some embodiments, the shell and/or the modified noble metal nanoparticle of the invention consists essentially of the conductive noble metal core and the ligands, as described herein. In some embodiments, at least 80%, at least 90%, at least 93%, at least 95%, at least 97%, at least 99.9%, or 100% by weight and/or volume of the shell consist of the ligands bound thereto, including any range between.

In some embodiments, at least 80%, at least 90%, at least 93%, at least 95%, at least 97%, at least 99.9%, or 100% including any range between, by weight of the modified noble metal nanoparticle consist of the conductive core and the ligands bound thereto.

In some embodiments, a weight ratio between the conductive core and the shell within the modified noble metal nanoparticle is between 20:1 and 2:1, between 20:1 and 15:1, between 15:1 and 10:1, between 10:1 and 3:1, between 10:1 and 2:1, between 10:1 and 8:1, between 8:1 and 5:1, between 5:1 and 3:1, between 5:1 and 2:1, including any range between.

In some embodiments, the modified noble metal nanoparticle is characterized by a resistance of less than 500,000 kΩ, less than 100,000 kΩ, less than 50,000 kΩ, less than 20,000 kΩ, less than 10,000 kΩ, less than 5,000 kΩ, less than 1000 kΩ, less than 500 kΩ, less than 100 kΩ, including any range between, when measured at 20° C. In some embodiments, the noble metal nanoparticle is characterized by a resistance between 1 and 500,000 kΩ, between 1 and 100,000 kΩ, between 10 and 10,000 kΩ, between 100 and 1000 kΩ, between 50 and 500 kΩ, including any range between. In some embodiments, the resistance of the modified noble metal nanoparticles refers to the resistance of the sensing element of the invention.

### Ligands

In some embodiments, the modified noble metal nanoparticle of the invention comprises the noble metal core, wherein the outer portion of the core is bound to the first ligand and to the second ligand, wherein the first ligand and the second ligand are as disclosed herein. In some embodiments, the first ligand and the second ligand assemble to form a shell on top of the noble metal nanoparticle.

In another aspect, there is provided a composition comprising the noble metal nanoparticles of the invention. In some embodiments, the composition consists essentially of the noble metal nanoparticles of the invention. In some embodiments, at least 80%, at least 90%, at least 93%, at least 95%, at least 97%, at least 99.9%, or 100% including any range between, by weight of the composition consist of the noble metal nanoparticles of the invention.

In some embodiments, the first ligand comprises (i) a linear or branched C1-C10 mercaptoalkyl; and/or (ii) a linear or branched C1-C10 mercaptoalkyl-aryl, including any salt thereof. In some embodiments, the first ligand is attached to the noble metal nanoparticle via thiol group (i.e. —S— or —SH).

In some embodiments, the mercaptoalkyl comprises a C2-C10 alkyl. In some embodiments, C2-C10 alkyl is a branched or a linear alkyl (optionally substituted), and comprising between 2 and 10, between 2 and 5, between 2 and 3, between 2 and 4, between 4 and 6, between 6 and 8, between 8 and 10, carbon atoms, including any range or value therebetween. In some embodiments, the mercaptoalkyl comprises a C1-C6 alkyl. In some embodiments, C1-C10 mercaptoalkyl is a branched or a linear alkyl (optionally substituted), being between 1 and 6, between 1 and 5, between 1 and 2, between 2 and 4, between 4 and 6, between 2 and 6, between 1 and 3, between 3 and 6, between 1 and 4, between 3 and 5, between 5 and 6, carbon atoms long, including any range or value therebetween.

The term “mercaptoalkyl-aryl” encompasses mercaptoalkyl covalently bound to an aryl via the terminal carbon atom of the mercaptoalkyl. In some embodiments, the mercaptoalkyl-aryl comprises a mercaptoalkyl being between 1 and 6, between 1 and 5, between 1 and 2, between 2 and 4, between 4 and 6, between 2 and 6, between 1 and 3, between 3 and 6, between 1 and 4, between 3 and 5, between 5 and 6, carbon atoms long abound to an aryl. In some embodiments, the aryl comprises a single aromatic ring (i.e. phenyl), a fused, a bicyclic or a polycyclic ring; wherein any one of the single aromatic ring, fused, bicyclic or polycyclic ring is substituted or non-substituted. In some embodiments, the aryl comprises a fused, a bicyclic or a polycyclic ring, wherein a first ring is phenyl and at least one additional ring is selected from an aliphatic ring (e.g. all-carbon 3-8-membered ring optionally comprising one or more heteroatom, such as O, S, N and/or NH), an aromatic ring (e.g. phenyl) and a heteroaromatic ring (e.g. 5-6 membered heteroaromatic ring). In some embodiments, the aryl comprises a fused, a bicyclic or a polycyclic ring, wherein the first ring and/or the additional ring(s) is substituted or non-substituted.

In some embodiments, each of the first ligand and the second ligand comprises a single species or a plurality of chemically distinct species.

In some embodiments, the second ligand is represented by Formula 1:

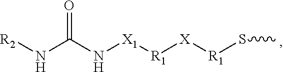

wherein a wavy bond represents H or an attachment point to the noble metal nanoparticle; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom and wherein R₂ represents an optionally substituted linear or branched alkyl, or H.

In some embodiments, the term “heteroatom” encompasses any one of O, OH, N, NR₁, N(R₁)₂, NH₂, S and SH, as allowed by valency. In some embodiments, the term “heteroatom” is or comprises O, OH, N, NH, NH₂, and S as allowed by valency. In some embodiments, the heteroatom is selected from the group consisting of O, S, NH, NRI, PH and PR₁ or a combination thereof; wherein R₁ is as described herein. In some embodiments, the heteroatom is O, NR₁ or S.

As used herein, the term “alkyl” describes an aliphatic hydrocarbon including straight chain and branched chain groups. In some embodiments, the alkyl group has 1 to 20 carbon atoms, between 1 and 10, between 1 and 5, between 5 and 10, between 10 and 15, between 15 and 20, including any range between.

In some embodiments, the term “alkyl”, as used herein, also encompasses saturated or unsaturated hydrocarbon, hence this term further encompasses alkenyl and alkynyl.

The term “alkenyl” describes an unsaturated alkyl, as defined herein, having between 6 and 30 carbon atoms and at least one carbon-carbon double bond. The alkenyl may be substituted or unsubstituted by one or more substituents, as described hereinabove. The term “alkynyl”, as defined herein, is an unsaturated alkyl having between 6 and 30 carbon atoms and at least one carbon-carbon triple bond. The alkynyl may be substituted or unsubstituted by one or more substituents, as described hereinabove.

In some embodiments, the term “cycloalkyl” describes an all-carbon monocyclic or fused ring (i.e., rings which share an adjacent pair of carbon atoms) group where one or more of the rings does not have a completely conjugated pi-electron system. The cycloalkyl may be substituted or unsubstituted, as indicated herein.

In some embodiments, the term “substituted” encompasses one or more (e.g., 2, 3, 4, 5, 6, or more) substituents, wherein the substituent(s) is as described herein.

In some embodiments, the second ligand is represented by Formula 1 including any salt and/or any tautomer thereof.

In some embodiments, the second ligand comprises a total number of carbon atoms within between 6 and 30, between 6 and 25, between 6 and 20, between 5 and 10, between 10 and 15, between 10 and 30, including any range or value between.

In some embodiments, a molar ratio between the first ligand and second ligand within the modified noble metal nanoparticles is between 5:95 and 95:5, between 5:95 and 1:15, between 5:95 and 1:10, between 5:95 and 1:5, between 5:95 and 1:1, between 1:15; and 95:5 and 10:1 and 95:5, between 1:5 and 95:5, between 1:1 and 95:5 and any range in between.

In some embodiments, the first and the second ligands are independently bound to the conductive core (e.g., via a coordinative bond) via a —SH or —S group (also used herein as “the binding moiety”).

In some embodiments, the binding moiety is or comprises thiol or thiolate.

In some embodiments, the modified noble metal nanoparticle of the invention comprises two ligand species.

In some embodiments, the first and second ligands are characterized by log P between 1.5 and 4, between 1.5 and 3.5, between 1.5 and 3, between 2 and 4, between 2 and 3.5, between 2 and 3, including any range between. P or partition coefficient is a compound specific value well-known in the art and refers to octanol-water partition coefficient. P can be determined experimentally, or alternatively can be predicted in-silico.

In some embodiments, the noble metal nanoparticles of the invention comprise a plurality of noble metal nanoparticles, wherein the noble metal nanoparticles differ from one another by the core (the noble metal type) and or the shell (the molar ratio between the first and second ligan).

In some embodiments, the modified noble metal nanoparticle of the invention is characterized by sensitivity to the analyte of interest, wherein sensitivity is as described herein. In some embodiments, the modified noble metal nanoparticle of the invention is characterized by enhanced binding affinity for the analyte of interest, as compared to a control (e.g., a VC or a VOC). In some embodiments, the binding affinity of the modified noble metal nanoparticle for the analyte of interest is enhanced by at least 2 times, at least 10 times, at least 100 times, at least 1000 times, at least 10.000 times, at least 1000.000 times, including any range between, as compared to a binding affinity for a control. In some embodiments, the modified noble metal nanoparticle of the invention is characterized by enhanced binding affinity for the analyte of interest, as compared to a control, wherein a concentration of the analyte of interest and of the control within the sample is substantially the same (e.g., less than 1000 ppm, less than 100 ppm, less than 10 ppm, less than 1 ppm, or between 0.01 ppb and 10,000 ppm, including any range between).

In some embodiments, the term “enhanced binding affinity” refers to an affinity ratio between (i) the binding affinity of the nanoparticle for the analyte of interest and (ii) the binding affinity of the nanoparticle for the control. In some embodiments, the enhanced binding affinity refers to an enhanced change in the electrical resistance of the nanoparticle upon exposure to the analyte of interest, as compared to the control. The binding affinity for a specific analyte may be deduced from the response intensity of a sensor (e.g., the sensor of the invention) upon exposure thereof to the specific analyte, wherein the sensor comprises the modified noble metal nanoparticles of the invention within the sensing element. For example, an enhanced binding affinity of the modified noble metal nanoparticle for a specific analyte will result in an enhanced response of the sensor. Thus, the affinity ratio of the modified noble metal nanoparticle, predetermines the selectivity and/or sensitivity of the sensor of the invention.

In some embodiments, the term “response” refers to a signal generated by the sensor in response to the conductivity change of the sensing unit.

In some embodiments, the control refers to any gas (e.g., a VC such as N₂, water, air, H₂ or a noble gas such as Ar), a mixture of gases and/or a VOC other than the analyte of interest. In some embodiments, a concentration of the control within a given gaseous sample is substantially the same or higher as the concentration of the analyte of interest. For example: a concentration of N₂, or Ar within a gaseous sample may be about 99.9%, and a concentration of an aldehyde (an exemplary analyte of interest) within a gaseous sample may be between about 0.1 ppb to 500 ppm, including any range between.

In some embodiments, the control refers to a VOC (e.g., an organic molecule having a chemical structure similar to the analyte of interest). In some embodiments, the control refers to a VOC present within a gaseous sample at a similar concertation range as the analyte of interest (e.g., between 0.01 ppb and 500 ppm, or more). In some embodiments, the control comprises, but is not limited to: isoprene, ketones (e.g. acetone), alcohols (e.g. methanol and/or ethanol), and water or any combination thereof. In some embodiments, a concentration of the control within a given gaseous sample is substantially the same or higher as the concentration of the analyte of interest. For example: a concentration of acetone within a gaseous sample may be between 300 and 800 ppb, a concentration of isoprene within a gaseous sample may be between about 100 ppb, a concentration of water within a gaseous sample may be about 1% (or 10,000 ppm), a concentration of an aldehyde (an exemplary analyte of interest) within a gaseous sample may be between about 3 to 100 ppb, including any range between.

In some embodiments, the control refers to a VOC (e.g., an organic molecule having a chemical structure similar to the analyte of interest). In some embodiments, the control refers to a VOC present within a gaseous sample at a similar concertation range as the analyte of interest (e.g., between 0.1 ppb and 1 ppm).

In another aspect, there is provided a chemiresistor sensor (e.g., sensor 100) comprising:
at least two electrodes; and at least one sensing element electrically connected to the two electrodes and comprising a deposited structure made from (or comprising) a plurality of modified noble metal nanoparticles of the invention, and wherein the chemiresistor sensor is configured to detect one or more analyte(s) of interest in a gaseous sample.
In some embodiments, the deposited structure is a pattern (e.g. a printed pattern) comprising the composition of the invention. In some embodiments, the structure is obtained via printing, spraying, applying, the composition of the invention on substrate (e.g., substrate 105).

In some embodiments, the sensor is configured to detect the presence of the analyte of interest and the concentration thereof in a gaseous sample. In some embodiments, the gaseous sample comprises the analyte of interest and the control. In some embodiments, the analyte of interest is a VOC.

As used herein, the term VOC refers to organic small molecules (usually having a molecular weight less than 1000 Da, or less than 500 Da) characterized by high vapor pressure (e.g., at least 10⁻¹⁰ atm) at room temperature.

In some embodiments, the analyte of interest is selected from or comprises an aldehyde, an alkane, or a combination thereof.

In some embodiments, the analyte of interest is water vapor. In some embodiments, the sensor is characterized by affinity to water vapors. In some embodiments, the sensor is characterized by an ability to detect the analyte of interest in the presence of water vapors in the gaseous sample.

In some embodiments, the plurality of modified noble metal nanoparticles of the invention comprises substantially the same particles. In some embodiments, at least 80%, at least 90%, at least 95%, at least 97%, at least 99%, including any range between, by total weight of the modified noble metal nanoparticles are chemically identical particles.

In some embodiments, at least a portion of the plurality of modified noble metal nanoparticles of the invention are chemically distinct particles (e.g., having chemically identical cores and chemically distinct specie(s) of the first ligand/and/or second ligand bound thereto). In some embodiments, the sensor comprises a plurality (e.g., 2, 3, 4, 5, 6 or 10 including any range between) of chemically distinct particle species.

In some embodiments, each of the modified noble metal nanoparticles of the invention comprises the same conductive core, and the same ligand ratio, wherein ligand ratio refers to the ratio between the first ligand and the second ligand with the shell. In some embodiments, the composition and/or the sensor of the invention comprises the modified noble metal nanoparticles of the invention characterized by differential ligand ratio within the shell. In some embodiments, the term “ligand ratio” refers to the ratio between the first ligand and second ligand contacting the noble metal nanoparticles core.

In some embodiments, the composition and/or the sensor of the invention comprises a first modified noble metal NP and a second modified noble metal NP, wherein each of first and second modified noble metal NP is independently characterized by the ligand ratio between 1:10 and 10:1, between 1:10 and 1:5, between 1:1 and 1:1, between 1:1 and 10:1, between 5:1 and 10:1, between 1:1 and 5:1 and 5:1 and 1:1, and any range in between.

In some embodiments, the average cross section size of the conductive core of the plurality of modified noble metal nanoparticles of the invention is between 1 and 100 nm, between 1 and 10 nm, between 10 and 50 nm, between 50 and 100 nm, including any range between. In some embodiments, the average cross section size of the conductive core is between 1 and 10 nm 1 and 5 nm, between 1 and 3 nm, between 3 and 5 nm, between 5 and 7 nm, between 7 and 10 nm, including any range between.

In some embodiments, the plurality of modified noble metal nanoparticles of the invention is characterized by a particle size in a range between 1 and 100 nm, between 1 and 10 nm, between 10 and 50 nm, between 50 and 100 nm, between 1 and 5, between 5 and 10 including any range between. In some embodiments, the modified noble metal nanoparticles of the invention are characterized by a particle size in a range between 1 and 5 nm, between 1 and 3 nm, between 3 and 5 nm, between 5 and 7 nm, between 7 and 10 nm, including any range between. In some embodiments, the term “particle size” refers to average cross section size of the modified noble metal nanoparticles.

In some embodiments, the plurality of modified noble metal nanoparticles is bound to a substrate. In some embodiments, each modified noble metal nanoparticle is bond to the substrate via a non-covalent bond (such as hydrogen bonds, electrostatic interactions, Van-der-Waals bonds, dipol-dipol interactions, etc.). In some embodiments, the plurality of modified noble metal nanoparticles are chemisorbed and/or physisorbed to the substrate. In some embodiments, the plurality of modified noble metal nanoparticles are stably bound to the substrate. In some embodiments, the term “stable” refers to the physical and/or chemical stability of the sensor unit (e.g., substantially devoid disintegration, or devoid of detachment of the modified particles from the sensing element) under operable conditions (a temperature of less than 100° C., normal pressure or vacuum, and optionally ambient atmosphere), for a time period of between 1 day and 1 year including any range between. In some embodiments, the stable sensor substantially maintains its affinity, selectivity and/or sensitivity to the analyte of interest.

In some embodiments, the analyte of interest comprises any of: an aldehyde, an aromatic compound, an alkane, an amine, a carboxylic acid or an ester thereof, an ether, a lactone, an alcohol, a phenol, ammonia, urea, silane, chlorinated alkane, chlorinated alkene, NOₓ, CO, HCl, CO₂, H₂, H₂S, O₂, H₂O, or any combination thereof.

In some embodiments, the analyte of interest comprises any of: an aldehyde, an optionally unsaturated aliphatic aldehyde or an aliphatic ketone, such as C1-C20, or C5-C20 aliphatic aldehyde or ketone, an optionally unsaturated alkane such as C1-C20, or C5-C20 alkane C6-C20 alkane C6-C10 alkane C5-C15 alkane. In some embodiments, the analyte of interest comprises a saturated alkane, optionally substituted by one or more substituents. In some embodiments, the saturated alkane comprises at least 2, at least 3, at least 4, at least 6, carbon atoms. In some embodiments, the saturated alkane comprises between C5-C20 C6-C20 C6-C10 and C5-C15 carbon atoms, including any range between.

In some embodiments, the analyte of interest comprises an aldehyde, wherein the aldehyde comprises a hydrocarbon chain comprising between 1 and 20 carbon atoms. In some embodiments, the aldehyde is an aliphatic aldehyde comprising at least 2, at least 3, at least 4, at least 6, or at least 5 carbon atoms. In some embodiments, the aldehyde is (C1-C20)-C(═O)—H.

In some embodiments, the modified noble metal nanoparticle(s) of the invention and/or the chemiresistor sensor comprising thereof, is characterized by an enhanced sensitivity to the gaseous analyte of interest, as compared to a control (e.g. a VOC such as a conjugated alkene (e.g. isoprene or any structurally related compound), a ketone (e.g. acetone), an alcohol and/or a VC such as N₂, CO₂, water, etc.), wherein the analyte of interest and the control are as described herein.

In some embodiments, the chemiresistor sensor is characterized by a porosity between 5 and 90%, between 5 and 15%, between 10 and 20%, between 15 and 45%, between 30 and 40%, between 40 and 60%, between 50 and 70%, between 70 and 90% and between 50 and 90% including any range between.

In some embodiments, the chemiresistor sensor is characterized by sensitivity and/or binding affinity to the analyte of interest, wherein sensitivity and binding affinity are as described herein.

In some embodiments, the sensitivity of the chemiresistor sensor to the analyte of interest is at least 2, at least 5, at least 10, at least 100, at least 1000, at least 10.000, at least 100.000, including any range between, as compared to the control. In some embodiments, the sensitivity of the chemiresistor sensor to the analyte of interest is enhanced by at least 5 times, at least 10 times, at least 100 times, at least 1000 times, at least 10.000 times, at least 100.000 times, including any range between, as compared to the control.

In some embodiments, the chemiresistor sensor is characterized by sensitivity and/or binding affinity to the analyte of interest under exposure to humidity. In some embodiments, the chemiresistor sensor is operable at a relative humidity of up to 40%, up to 50%, up to 60%, up to 70%, up to 80%, up to 85% or up to 90%, including any range between. In some embodiments, the chemiresistor sensor is capable of detecting one or more analytes of interest within a gaseous sample, wherein the gaseous sample further comprises up to 40%, up to 50%, up to 60%, up to 70%, up to 80%, up to 85%, or up to 90% water vapors.

In some embodiments, the sensitivity of the chemiresistor sensor to the analyte of interest in a relative humid condition is enhanced by at least 5 times, at least 10 times, at least 100 times, at least 1000 times, at least 10.000 times, at least 100.000 times, including any range between, as compared to the control. As used herein the term “sensitivity” refers to the ratio between signal intensities of the sensor in response to the analyte of interest and in response to the control (e.g., being referred to as 1), wherein the control and the analyte of interest are present within the sample.

Accordingly, the term “enhanced” when referring to sensitivity relates to the signal enhancement in response to the analyte of interest, relative to the response of the sensor to the control (wherein the signal intensity in response to the control is optionally referred to as 1). In some embodiments, one or analyte of interest and the control are present within the sample at the same concentration range (e.g., at sub-ppb and up to ppm range). In some embodiments, the term “sensitivity” refers to a sample comprising a concentration of the control and of the analyte of interest ranging between 0.01 ppb and 500 ppm, between 0.1 ppb and 500 ppm, between 0.1 ppb and 10.000 ppm, including any range between. In some embodiments, the sensitivity of the sensor is predetermined by the affinity ratio of the nanoparticle of the invention. In some embodiments, the sensor is characterized by a detection limit (LOD) of at least 0.1 ppb or at least 0.05 ppb. In some embodiments, the sensor is characterized by a detection limit (LOD) of between 0.01 ppb and 500 ppm, between 0.1 ppb and 500 ppm, between 0.5 ppb and 500 ppm, between 0.5 ppb and 100 ppm, between 0.1 ppb and 100 ppm, between 0.1 ppb and 10 ppm, including any range between. A skilled artisan will appreciate that the LOD may vary for each specific analyte.

In some embodiments, the sensor is configured to selectively detect the analyte of interest within the sample. In some embodiments, the sensor is configured to selectively detect the analyte of interest, wherein a concentration of the analyte of interest within the sample is 0.01 ppb and 10000 ppm, or between 0.1 ppb and 10000 ppm, including any range between.

In some embodiments, the sensor may be included in an array of sensing elements configured to detect the presence and/or concentration of an analyte of interest, for example, an aldehyde and/or an alkane, as described herein.

In some embodiments, the sensor comprises residual amount of an organic solvent, wherein the organic solvent is as disclosed in the Method section below. In some embodiments, the residual amount is in a range between 0.1 ppb and 1 ppm, between 0.1 ppb and 0.1 ppm, between 0.1 ppm and 0.5 ppm, between 0.1 and 1 and between 0.5 and 1 ppm, including any range between.

### Method for Detection of Analyte

In another aspect, there is provided a method for detection of an analyte of interest in a gaseous sample, comprising:
a. exposing the sensor of the invention to the gaseous sample;
b. providing electricity to the sensor, so as to obtain a plurality of values generated by the sensor; and
c. analyzing the values, thereby determining the presence and level of intensity (or concentration) of the analyte of interest within the gaseous sample. In some embodiments, wherein the analyte of interest is as described hereinabove. In some embodiments, the analyte of interest comprises a plurality of analytes.

In some embodiments, the plurality of values comprises at least one of: conductivity, resistivity, capacitance, impedance, current and voltage of the sensor.

In some embodiments, providing electricity may include, measuring at least one value selected from conductivity, resistance, capacity, impedance, current and voltage of the sensor, when the sensor is exposed to the analyte of interest.

Upon exposing the sensor to the presence of analyte, the resistivity of the sensing element changes. In some embodiments, measuring values related to the resistivity, conductivity, capacity and/or impedance of the at least one sensing element between the two electrodes, prior, during and after the exposure to the analyte is indicative to the amount and/or type of analyte attached/engaged with the sensing element. In some embodiments, the measured values are selected from: a temporal resistance, temporal conductivity, base resistance (e.g., background resistance), base conductivity, electrical noise, base current, base voltage, base frequency, and base amplitude.

In some embodiments, the measured values may further be analyzed and manipulated (by a processor associated with the chemiresistor sensor) in order to further extract additional values. In some embodiments, the extracted values are selected from: the maximal subtracted resistance, the difference between maximum and minimum values, the difference between maximum and minimum values divided by base resistance (normalized response), the average value, the maximum value, the minimum value, the first time derivative, the second time derivative, signal to noise ratio, incline gradient, decline gradient, rise time, overshooting value relative to steady state value, oscillation decay in time and oscillation frequency.

In some embodiments, the measured values and/or the extracted values may be used for detecting the presence and/or amount of a specific analyte, for example, aldehydes. In some embodiments, the measured values and/or the extracted values may be compared to a prestored data, for example, using any mathematical correlation and/or a calibration curve. In some embodiments, the mathematical correlation is one of: a linear correlation, a parabolic correlation, a polynomial correlation, logarithmic correlation, exponential correlation and power correlation.

In some embodiments, the sample comprises a plurality of gaseous analytes. In some embodiments, the sample comprises a plurality of VOCs. In some embodiments, the sample comprises one or more analytes of interest (e.g., an aldehyde, an optionally unsaturated aliphatic aldehyde such as C1-C20, or C5-C20 aliphatic aldehyde, an optionally unsaturated alkane such as C1-C20, C6-C20 alkane or C5-C20 alkane, or any combination thereof) and at least one additional gaseous species selected from alkene, alcohol, water and ketone or any combination thereof. In some embodiments, the sample further comprises one or more atmospheric gases (e.g., N₂, O₂, CO₂, and/or water). In some embodiments, the sample has a temperature between −10 and 100° C., between −10 and 50° C., between 0 and 30° C., between 0 and 40° C., between 50 and 100° C., including any range between.

In some embodiments, the method is for selectively detecting the presence of the analyte of interest within the sample, wherein a concentration of the analyte within the sample is at least 0.01 ppb, at least 0.05 ppb, at least 0.1 ppb, at least 1 ppb, at least 10 ppb, at least 50 ppb, at least 100 ppb, at least 500 ppb, including any range between. The inventors implemented exemplary nanoparticles of the invention for detection of selected VOCs at a concentration about 0.1 ppb, or less.

In some embodiments, the gaseous sample comprises the analyte of interest at a concentration between 0.1 and 500 ppb, between 0.1 ppb and 500 ppm, or between 1 ppb and 1 ppm; and a concentration of the additional species is at most 10 ppm, at most 1 ppm, at most 500 ppb, at most 100 ppb, at most 1 ppm, at most 10 ppm, at most 100 ppm, at most 500 ppm, and wherein the additional species is as described herein.

In some embodiments, exposing step is performed under operable conditions. In some embodiments, operable conditions comprise (i) a contacting time of at least 1 second (s), at least 2 s, at least 5 s, at least 8 s, at least 10 s, at least 30 s, at least 60 s, at least 1.5 minute (min), at least 2 min, at least 3 min, at least 5 min, or between 10 s and 10 min, between 10 s and 60 s, between 1 and 2 min, between 1 and 10 min, between 2 and 5 min, between 5 and 10 min, including any range between; and (ii) an operable temperature between −10 and 100° C., between 0 and 10° C., between 0 and 25° C., between 10 and 30° C., between 10 and 20° C., between 10 and 40° C., between 20 and 30° C., between 30 and 40° C., between 40 and 50° C., including any range between.

In some embodiments, operable conditions comprise a pressure between 0.5 and 2 bar, a relative humidity of between 0 and 100%, between 10 and 100%, between 0 and 10%, between 10 and 30%, between 30 and 100%, between 40 and 100%, between 40 and 60%, between 40 and 70%, between 40 and 80%, between 60 and 80%, between 80 and 100%, including any range between.

In some embodiments, exposing comprises contacting the chemiresistor sensor with a flowing gaseous sample. In some embodiments, the flowing gaseous sample is characterized by a flow rate between 50 and 2000 ml/min, between 50 and 100 ml/min, between 100 and 200 ml/min, between 100 and 2000 ml/min, between 200 and 1000 ml/min, between 200 and 500 ml/min, between 500 and 2000 ml/min, between 500 and 1000 ml/min, between 1000 and 2000 ml/min, including any range between.

### Method for Preparing the Modified Noble Metal Nanoparticles

In some embodiments, there is provided a method for manufacturing the modified noble metal nanoparticles of the innovation, the method comprises (i) contacting the noble metal nanoparticles bound to the first ligand of the invention (NNP) with a compound of Formula 2:

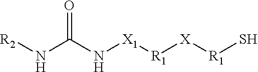

under suitable conditions, thereby obtaining the modified noble metal nanoparticles; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom; and wherein R₂ represents an optionally substituted linear or branched alkyl. In some embodiments, suitable conditions are sufficient for performing at least partial ligand exchange, thereby obtaining the modified noble metal nanoparticles, wherein each modified noble metal nanoparticle is bound to the first ligand and to the second ligand. In some embodiments, suitable conditions are sufficient for performing at least partial ligand exchange, thereby obtaining a plurality of the modified noble metal nanoparticles, wherein a w/w or molar ratio between the first ligand and the second ligand bound to the plurality of the modified noble metal nanoparticles is as disclosed hereinabove.

In some embodiments, the contacting step (i) is performed in a solution. In some embodiments, the contacting step (i) comprises contacting noble metal NPs with a solution comprising the compound of Formula 2. In some embodiments, the solution comprises a polar organic solvent suitable for dissolving the compound of Formula 2. The organic solvent and the suitable conditions are as described herein below. In some embodiments, the noble metal NPs are Au NPs.

In some embodiments, the contacting step (i) is performed for a time period (and/or under conditions) sufficient for obtaining at least 5%, at least 10%, at least 20%, between 5 and 60%, between 5 and 30%, between 5 and 20%, between 5 and 90%, between 5 and 80%, between 10 and 80%, between 10 and 50% ligand exchange (also used herein as “conversion”), including any range between. In some embodiments, the conversion of the contacting step (i) is monitored by any known analytical method, such as XRD, NMR, XPS, TGA, elemental analysis, etc. A skilled artisan will appreciate the exact analytical method for monitoring the conversion and thus determining the time period when the contacting step (i) is completed (e.g., the point when the conversion graph reaches a plateau or a steady state).

In some embodiments, noble metal NPs are bound to a substrate. In some embodiments, contacting step (i) comprises contacting the noble metal NPs bound to a substrate with the compound of Formula 2. In some embodiments, contacting step (i) comprises contacting the noble metal NPs bound to a substrate with a solution comprising the compound of Formula 2.

In some embodiments, there is provided a method for manufacturing the modified noble metal nanoparticles of the innovation, the method comprises (i) contacting the noble metal NPs bound to a substrate with the compound of Formula 2 under suitable conditions, thereby obtaining the modified noble metal nano particles of the invention bound to the substrate. In some embodiments, the method comprises a preliminary step of bonding the noble metal NPs to the substrate, thereby obtaining the noble metal NPs bound to the substrate.

In some embodiments, there is provided a method for manufacturing the modified noble metal nanoparticles of the innovation, the method comprises bonding the noble metal NPs to the substrate, thereby obtaining the noble metal NPs bound to the substrate (ii) performing a ligand exchange by reacting the noble metal NPs bound to the substrate with the compound of Formula 2 under conditions suitable for ligand exchange, thereby obtaining the modified noble metal nano particles of the invention bound to the substrate.

In some embodiments, the method of the invention further comprises a step of washing the modified noble metal nano particles of the invention (unbound modified noble metal nano particles, or the modified noble metal nano particles bound to the substrate), thereby removing unbound compound of Formula 2 and/or solvent of the ligand exchange step from the modified noble metal nano particles. In some embodiments, washing is performed by contacting the modified noble metal nano particles with a sufficient amount of an organic solvent. In some embodiments, washing is performed subsequent to the ligand exchange step.

In some embodiments, the method further comprises (iii) drying the modified noble metal nano particles (unbound modified noble metal nano particles, or the modified noble metal nano particles bound to the substrate), thereby obtaining dried modified noble metal nano particles (dry NoNP).

In some embodiments, there is provided a method for manufacturing the modified noble metal nanoparticles of the innovation, the method comprises bonding the NNPs to the substrate, thereby obtaining the NNPs bound to the substrate (ii) performing a ligand exchange by reacting the NNPs bound to the substrate with the compound of Formula 2 under conditions suitable for ligand exchange, thereby obtaining the modified noble metal nano particles of the invention bound to the substrate; (iii) drying the modified noble metal nanoparticles bound to the substrate, thereby obtaining dried modified noble metal nano particles (dry MoNP).

The terms “contacting step” and “ligand exchange” are used herein interchangeably.

In some embodiments, the bonding step is performed by printing.

In some embodiments, contacting step (i) is performed in a polar organic solvent (e.g., toluene, chloroform etc.) suitable for dissolving the compound of Formula 2. In some embodiments, the solubility of the compound of Formula 2 within the polar organic solvent is at least 0.01 g/L, at least 0.1 g/L, at least 0.5 g/L, at least 1 g/L, at least 5 g/L, including any range between.

In some embodiments, the polar organic solvent is selected from ketones (e.g., acetone), alcohols (e.g., ethanol and methanol), nitrile (e.g., acetonitrile and butyronitrile), amide (e.g., DMF), aromatic (e.g., toluene, xylene), esters (e.g., ethyl acetate) and haloalkanes (e.g., chloroform and dichloromethane), including any combination thereof.

In some embodiments, suitable conditions for the ligand exchange step comprise a temperature between −20 to 60° C., between −20 to −10° C., between −10 to 0° C., between 0 to 10° C., between 10 to 20° C., between 20 to 30° C., between 30 to 40° C., between 40 to 50° C., between 50 to 60° C. including any range in between. In some embodiments, the reaction temperature of the ligand exchange is between 0 and 70° C., between 0 and 10° C., between 10 and 20° C., between 20 and 25° C., between 20 and 30° C., between 40 and 50° C. and between 50 and 70° C., including any range in between.

In some embodiments, the drying step is performed by convention drying methods, such as thermal drying, vacuum drying, or both.

In some embodiments, drying comprises exposing the MoNPs, or the MoNPs bound to the substrate to a temperature between 20 to 60° C., between 20 to 25° C., between 25 to 30° C., between 30 to 35° C., between 35 to 40° C., between 40 to 45° C., between 45 to 50° C., and between 50 to 60° C., including any range in between.

In some embodiments, drying is performed at a normal pressure (about 1 atm).

In some embodiments, steps (i) to (iii) are preformed sequentially.

In some embodiments, dry MoNP comprise trace amounts of an organic solvent. In some embodiments, dry MoNP comprise trace amounts of the organic polar solvent, as disclosed herein. In some embodiments, dry MoNP comprise trace amounts of the organic polar solvent ranging between 0.1 ppb and 1 ppm, between 0.1 ppb and 0.1 ppm, between 0.1 ppm and 0.5 ppm, between 0.1 and 1 and between 0.5 and 1 ppm, including any range in between.

In some embodiments, suitable conditions for the ligand exchange step comprise a reaction time of at least 0.5 hour (h), at least 1 h, at least 2, at least 3 h, at least 4 h, at least 5 h, at least 8 h, at least 16, at least 20, at least 40, at least 48 h, between 0.5 and 48 h, including any value or range in between.

In some embodiments, bonding is performed by printing an ink comprising the NNPs dispersed therewithin. In some embodiments, the ink is manufactured by dispersing the NNPs within an organic solvent, by methods such as, sonication, ultrasonic methods, homogenizing stirring method (e.g., using blades) and the like.

In some embodiments, the NNPs are dispersed in a solvent mixture (e.g., toluene, ethanol, acetone, other—ketones, alcohols, aromatic, or the like) to form a stable ink, or possibly paste depending on printing method. The printing methods may be selected from: inkjet, die coating, screen printing, Dr, blade printing, or any other three-dimensional deposition method.

In some embodiments, ink is applied (by printer) to form the sensing element (e.g., sensing element 130) at a specific pattern on the surface of the substrate (e.g., Si-substrate) and/or on the surface of the electrode. Nonlimiting example for the size of such sensing element are, 1 mm×1 mm and a thickness of between 100 nm to 3000 nm. In some embodiments, the thickness is smaller than 3000 nm. In some embodiments, the thickness is smaller than 2000 nm. In some embodiments, the thickness is smaller than 1000 nm. In some embodiments, the thickness is smaller than 500 nm. In some embodiments, the thickness is smaller than 400 nm. In some embodiments, the thickness is smaller than 200 nm. In some embodiments, the thickness is smaller than 100 nm. At the end of the deposition/printing process solvents residues are extracted from the sensing element by natural evaporation or heat application (oven, light, UV, other), or vacuum application or combination.

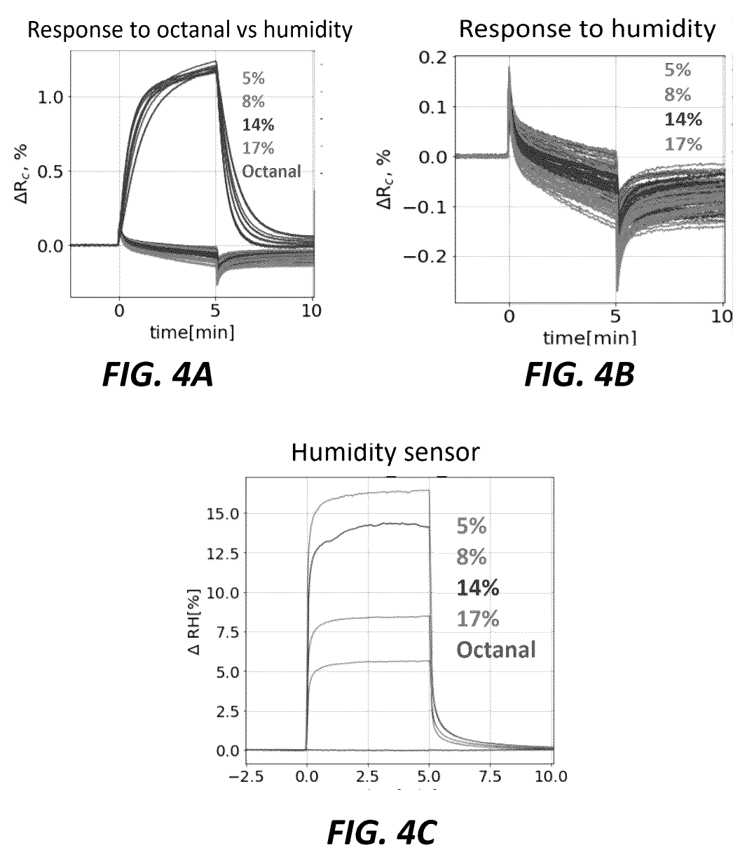

Reference is now made to FIG. 3A which is a flowchart of a method of making modified noble metal nanoparticles according to some embodiments of the invention. The method comprises contacting the noble metal nanoparticles bound to the first ligand of the invention with a compound of Formula 2 (step 400), thereby obtaining the modified noble metal nano particles (step 402). Second, contacting the modified noble metal nanoparticles with a substrate (step 404), thereby obtaining the modified noble nanoparticles bound to the substrate (step 406).

Reference is now made to FIG. 3B which is a flowchart of an exemplary method of manufacturing of modified noble metal nanoparticles according to some embodiments of the invention. The method comprises, providing the noble metal NPs and the substrate (step 420) and bonding the noble metal NPs to the substrate, thereby obtaining the noble metal NPs bound to the substrate (step 422). And subsequently, performing a ligand exchange by reacting the noble metal NPs bound to the substrate with the compound of Formula 2 under conditions suitable for ligand exchange (step 424), thereby obtaining the modified noble metal nano particles of the invention bound to the substrate (step 426).

In some embodiments, there is provided a method for manufacturing the modified noble metal nanoparticles of the innovation, the method comprises bonding the NNPs to the substrate, thereby obtaining the NNPs bound to the substrate (ii) performing a ligand exchange by reacting the NNPs bound to the substrate with the compound of Formula 2 under conditions suitable for ligand exchange, thereby obtaining the modified noble metal nano particles of the invention bound to the substrate; and (iii) drying the modified noble metal nanoparticles bound to the substrate, thereby obtaining dried modified noble metal nano particles (dry MoNP).

In some embodiments, the sensing element comprises a substrate in contact with the plurality of nanoparticles of the invention, wherein at least 50%, at least 70%, at least 90%, at least 95%, at least 97%, at least 99%, at least 99.9% of at least one substrate surface is in contact with or covered by the nanoparticles of the invention. In some embodiments, the nanoparticles of the invention form a substantially homogenous layer a uniform thickness. In some embodiments, the layer thickness is characterized by a standard deviation of about 10%, about 20%, about 50%, about 5%, about 1%, about 0.1%, about 0.01%, including any range between.

In some embodiments, the layer is a patterned layer, characterized by a plurality of areas, each area having a different thickness, wherein the thickness within the entire area of the pattern is substantially uniform. In some embodiments, the thickness of the patterned layer (or of a specific area within the patterned layer) ranges between 50 nm and 5 um, including any range between.

In some embodiments, the noble metal within the NNP is in a crystalline state, or in amorphous state. In some embodiments, a pristine noble metal NPs (i.e. nanoparticles devoid of the first and second ligand(s)) are prepared by well-known synthetic procedures, such as a co-dispersion, and/or two-phase reduction.

In some embodiments, the method of the invention further comprises a preliminary step of manufacturing the NNPs, wherein the preliminary step is performed prior to performing the step (i). In some embodiments, the preliminary step comprises contacting the pristine noble metal NPs with the first ligand of the invention under suitable conditions, thereby obtaining the NNPs.

In some embodiments, the preliminary step is performed in a solution. In some embodiments, the preliminary step comprises contacting noble metal NPs with a solution comprising the first ligand. In some embodiments, the solution comprises a polar organic solvent suitable for dissolving the first ligand.

In some embodiments, the preliminary step is performed for a time period (and/or under conditions) sufficient for binding at least 5%, at least 10%, at least 20%, between 5 and 60%, between 5 and 30%, between 5 and 20%, between 5 and 90%, between 5 and 80%, between 10 and 80%, between 10 and 50 mol % of the initial amount of the first ligand in the solution, including any range between (also referred to herein as “binding efficiency”). In some embodiments, the binding efficiency is monitored by any known analytical method, such as XRD, NMR, XPS, TGA, elemental analysis, etc. A skilled artisan will appreciate the exact analytical method for monitoring the binding efficiency and thus determining the time period when the preliminary step is completed (e.g., the point when the binding efficiency reaches a plateau or a steady state).

In some embodiments, the preliminary step further comprises washing, and/or drying the NNPs.

In some embodiments, suitable conditions for the preliminary step comprise (i) a reaction time of at least 1 minute (m), at least 10 m, at least 0.5 hour (h), at least 1 h, at least 2, at least 3 h, at least 4 h, at least 5 h, at least 8 h, at least 16, at least 20, at least 40, at least 48 h, between 1 m and 48 h, including any value or range in between; and (ii) a temperature between 0 and 100° C., or any temperature above the freezing point and below the boiling point of the solvent.

### General

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” refers to at least 80%, at least 85%, at least 90%, at least 92%, at least 95%, at least 96%, at least 97%, at least 98%, at least 99%, including any range or value therebetween. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and/or engineering arts.

Further, all numerical values, e.g., when referring the amounts or ranges of the elements constituting the formulation are approximations which are varied (+) or (−) by up to 20%, at times by up to 10% of from the stated values. It is to be understood, even if not always explicitly stated that all numerical designations are preceded by the term “about”.

The term “consisting essentially of” is used to define formulations which include the recited elements but exclude other elements that may have an essential significance on the formulation. “Consisting of” shall thus mean excluding more than trace elements of other elements. Embodiments defined by each of these transition terms are within the scope of this invention.

The term “substituent”, as used herein comprises one or more substituents (e.g. 1, 2, 3, 4, 5, or 6), each independently selected from the group consisting of: C₁-C₆ alkyl, halo, —NO₂, —CN, —OH, —NH₂, carbonyl, —CONH₂, —CONR′₂, —CNNR₂, —CSNR₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C₁-C₆ alkyl)₂, C₁-C₆ alkoxy, C₁-C₆ haloalkoxy, hydroxy(C1-C6 alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R′, —OCOR′, —OCOR′, —OC(═O)OR′, —OC(═O)NR′, —OC(═S)OR′, —OC(═S)NR′, a heteroatom, an optionally substituted cycloalkyl, an optionally substituted heterocyclyl, or a combination thereof, wherein each R′ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom).

As used herein, the term “alkyl” describes an aliphatic hydrocarbon including straight chain and branched chain groups. In some embodiments, the alkyl group has 1 to 20 carbon atoms, between 1 and 10, between 1 and 5, between 5 and 10, between 10 and 15, between 15 and 20, including any range between.

In some embodiments, the alkyl encompasses a short alkyl and/or along alkyl. In some embodiments, the alkyl has from 21 to 100 carbon atoms, or more. In the context of the present invention, a “long alkyl” is an alkyl having at least 20 carbon atoms in its main chain (the longest path of continuous covalently attached atoms). A short alkyl therefore has 20 or less (e.g., 2, 3, 4, 5, 6, 8, 10, 15, or 20) main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulphur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

In some embodiments, R′ and R″ are the same or different, wherein each of R′ and R″ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′. A “nitro” group refers to a —NO₂ group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —RNC(N)NR″R′″ end group or a —R′NC(N) NR″— linking group, as these phrases are defined hereinabove, where R′, R″ and R′″ are as defined herein. As used herein, the term “azide” refers to a —N₃ group. The term “sulfonamide” refers to a —S(O)₂NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C1-6-alkyl, C1-6-alkyl, hydroxy-C1-6-alkyl and amino-C1-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

### Examples

The inventors successfully manufactured sensors that are operable at a relative humidity of up to 90%, as opposed to the currently available chemiresistor sensors being characterized by a varying response depending on the water content of the gaseous sample.

### Sensor Preparation

### Method 1:

Gold nanoparticles capped with the first ligand are deposited on a chip and the chip is dipped into a solution with the second ligand. After between 1 and 20 h the chip is rinsed and dried using a vacuum chamber. The sensing element performance depends on the time and temperature of the ligand exchange reaction.

### Method 2:

Gold nanoparticles capped with the first ligand dispersed in a solvent (1-20 mg/mL) are mixed with the second ligand (5-100 mM). The dispersion is mixed over a period of 1-20 h. Then the nanoparticles capped with the first and the second ligands are isolated and washed using centrifuge. The resulted NP powder is dispersed in a mixture of solvents that compose ink for deposition onto the substrate. The ink and sensing element performance depends on a time and temperature of ligand exchange reaction.

### Sensor Response

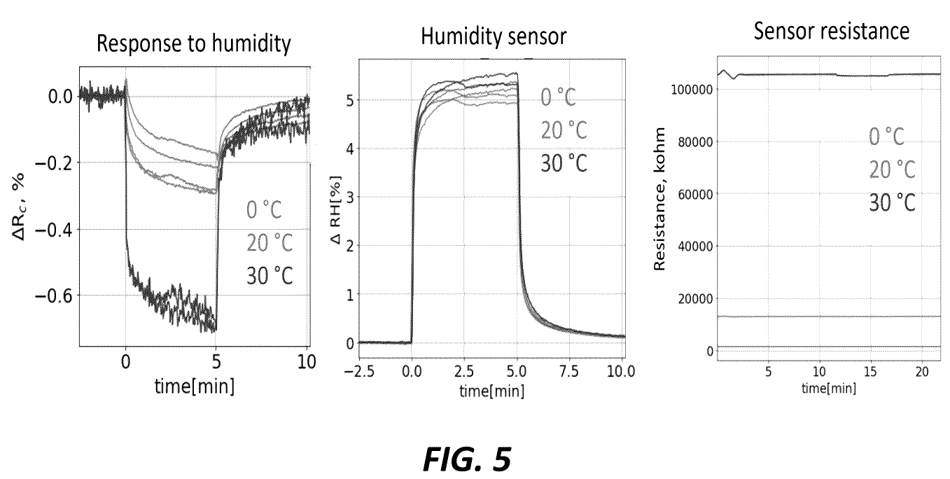

The sensor response was examined in different humidity percentages (5-17%). The inventors surprisingly observed that the response of the sensor to water vapor remained almost constantly near zero at variable water content in the sample (FIG. 4A-4C).

Furthermore, the sensor exhibited a significant sensitivity to octanal. The response to octanal was only slightly affected by adding 5% relative humidity.

### Effect of Ligand Ratio in the Shell on the Humidity Sensor

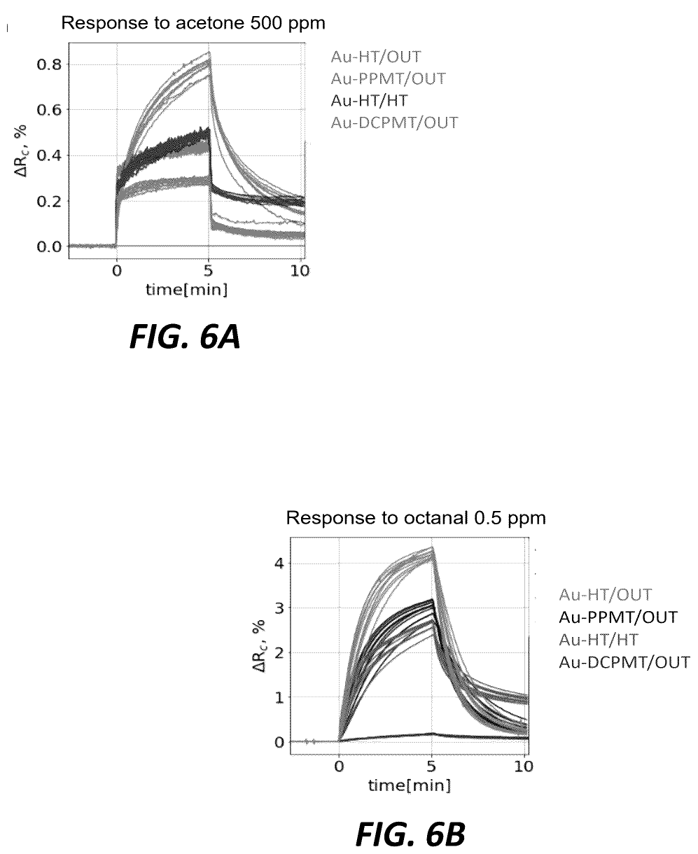

In order to evaluate the conversion rate of the ligand exchange as a function of the reaction temperature, ligand exchange reaction was performed at three different temperatures (0, 20 and 30°), and the response to octanal and 5% relative humidity was tested (FIG. 5). The increase in temperature increases the ligand exchange process, increasing the amount of the second ligand in the shell. The response to humidity is negative and it turns more negative with increased ligand exchange reaction temperature (FIG. 5).

X-ray diffraction analysis (XPS) demonstrated significant increase in N/C ratio from 0.02 to 0.075 and a decrease in the Au/C ratio from 1.01 to 0.07 upon the ligand exchange process. These results. support that part of the hexanethiol (HT) was replaced with 1-(tert-butyl)-3-((11-mercaptoundecyl)oxy)urea (OUT), obtaining the modified gold nano particles of the invention (Au-HT/OUT). HT comprises 0 nitrogen atoms and 6 carbon atoms whereas the OUT comprises of 2 nitrogen atoms and 16 carbon atoms.

XPS showed presence of Pt and Si (stemming from the substrate and electrode materials) before exchange of HT and no signal for Pt and Si after exchange with OUT. This might indicate that partial exchange of HT with OUT results in gold cores and ligands organization that blocks X-ray to reach chip and electrode surface. This observation can explain why Au-HT/OUT has no response to humidity and why its response to humidity is the same at different levels of humidity. It is postulated that the steady response of Au-HT/OUT based sensor of the invention at varying water contents of the sample is due to physical blocking of chip and electrode surface from being reached by humidity.

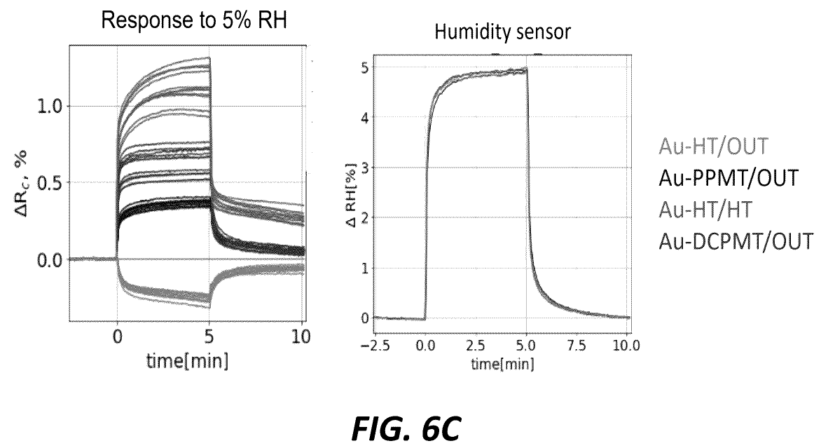

The inventors examined similar gold nanoparticles bound to alkyl-aryl thiols (PPMT and DCPT) as the first ligand, and further bound to OUT as the second ligand. The resulting nanoparticles were assigned as Au-PPMT/OUT and Au-DCPMT/OUT, respectively. Sensors were made from Au-PPMT/OUT or Au-DCPMT/OUT and their performance to 5% relative humidity (FIG. 6A), acetone (FIG. 6B) and octanal (FIG. 6C) was examined. Results are summarized in Table 1.

TABLE 1

| | Sensitivity to | Sensitivity to | Sensitivity to |
|---|---|---|---|
| First ligand | octanal | acetone | humidity |
| hexane thiol | ↑ | ↓ | ↓ |
| PPMT | ↑ | Didn't change | Didn't change |
| DCPMT | ↓ | ↓ | ↑ |

The sensor comprising Au-PPMT/OUT nanoparticles, demonstrated increased sensitivity to octanal and similar sensitivity to acetone and relative humidity compared to the control sensor, consisting of gold nanoparticles contacting only HT(hexane thiol) ligands.

The sensor comprising Au-DCPMT nanoparticles, demonstrated increase sensitivity to humidity and a decrease in sensitivity to acetone and octanal compared to the control sensor.

The sensor comprising Au-HT/OUT (exemplary sensor of the invention) demonstrated increase sensitivity to octanal and a decrease in sensitivity to acetone and relative humidity compared to the control sensor.

What is claimed is:

1. A composition comprising a plurality of modified noble metal nanoparticles, wherein each of said plurality of modified noble metal nanoparticles comprises a noble metal nanoparticle bound to a first ligand and to a second ligand, wherein:
the first ligand and the second ligand are assembled to form a shell on top of said noble metal nanoparticle;
said first ligand is a linear or branched C1-C10 mercaptoalkyl, or a linear or branched C1-C10 mercaptoalkyl-aryl;
said second ligand is represented by Formula: 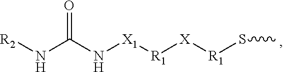
wherein a wavy bond represents H or an attachment point to said noble metal nanoparticle; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom; and wherein R₂ represents H, or an optionally substituted linear or branched alkyl.

2. The composition of claim 1, wherein a total number of carbon atoms within said second ligand is between 6 and 30.

3. The composition of claim 1, wherein a molar ratio between the first ligand and the second ligand within said composition is between 5:95 and 95:5.

4. The composition of claim 1, wherein said modified noble metal nanoparticle is characterized by sensitivity to an analyte of interest.

5. The composition of claim 4, wherein the analyte of interest comprises a volatile organic compound (VOC), water, nitrogen oxide (NOₓ), CO₂, ammonia, urea, H₂S, H₂, O₂, CO, sulfonated compounds, halogenated compounds, silane or any combination thereof.

6. The composition of claim 5, wherein the VOC is selected from an optionally unsaturated C1-C20 aldehyde, an optionally unsaturated C1-C20 ketone, an optionally unsaturated C1-C20 alkane, a chlorinated alkane an aromatic compound, carboxylic acid, ester, ether, lactone, alcohol, phenol-based compounds, or any combination thereof.

7. The composition of claim 1, wherein a total number of carbon atoms within said second ligand is between 10 and 30.

8. The composition of claim 1, wherein said plurality of modified noble metal nanoparticles is bound to a substrate.

9. The composition of claim 8, wherein said composition is characterized by a porosity between 5 and 90%.

10. A method for synthesizing the composition of claim 1, the method comprises reacting noble metal nanoparticles bound to the first ligand with a compound of Formula: 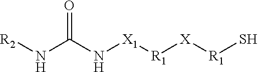
under suitable conditions, thereby obtaining said modified noble metal nanoparticles; wherein each R₁ independently represents an optionally substituted linear or branched alkyl optionally comprising one or more heteroatom(s), or an optionally substituted cycloalkyl optionally comprising one or more heteroatom(s); wherein X represents a bond or a heteroatom; wherein X₁ represents a bond, an optionally substituted linear or branched alkyl, or a heteroatom; and wherein R₂ represents an optionally substituted linear or branched alkyl.

11. The method of claim 10, wherein said conditions comprise a temperature between −20 and 60° C. and a reaction time of at least 0.5 h.

12. The method of claim 10, wherein said conditions comprise a polar organic solvent with a relative polarity of at least 0.15.

13. The method of claim 10, wherein said noble metal nanoparticles are bound to a substrate; and wherein said contacting comprises a molar ratio between said compound and said first ligand of between 2:1 and 1000:1, optionally wherein said method further comprises drying said noble metal nanoparticles.

14. A chemiresistor sensor comprising:
at least two electrodes; and
a sensing element electrically connected to the two electrodes and comprising a structure comprising the composition of claim 1.

15. The chemiresistor sensor of claim 14, wherein the distribution of the modified noble metal nanoparticles within the structure is such that the entire structure is electrically conductive; and wherein the modified noble metal nanoparticles are bound to a substrate comprising a plurality of electrodes; and wherein the modified noble metal nanoparticles are in a form of a substantially continuous layer on top of the substrate.

16. The chemiresistor sensor of claim 14, wherein the sensor is configured for selective detection of group of analytes of interest within a gaseous sample by reducing the response to other group of analyte, and wherein said sensor is characterized by a limit of detection (LOD) ranging between 0.01 ppb and 500 ppm.

17. The chemiresistor sensor of claim 14, wherein said sensor is operable at a relative humidity of up to 90%.

18. A method for detection of an analyte of interest in a gaseous sample, comprising:
a. exposing the chemiresistor sensor of claim 14 to the gaseous sample;
b. providing electricity to the sensor, so as to obtain a plurality of values generated by the sensor; and
c. analyzing said values thereby determining the presence of the analyte of interest within said sample, wherein said values comprise conductivity values, capacitance values, impedance values, or any combination thereof.

19. The method of claim 18; wherein said analyte of interest comprises a volatile organic compound (VOC), nitrogen oxide (NOₓ), CO₂, ammonia, H₂S, or any combination thereof; wherein said analyzing step further comprises determining a concentration of the analyte of interest within the sample.

20. The method of claim 18, wherein a concentration of the analyte of interest within the sample is between 0.1 ppb and 500 ppm.
